(19) United States
(12) Patent Application Publication
(10) Pub. No.: US 2026/0325390 A1
(43) Pub. Date: Oct. 22, 2026

(54) SELF-SUPPORTING UNIVERSAL MONITORING ASSEMBLY FOR ROLLATOR

(71) Applicant: Shenzhen Aeon Technology Co., Ltd., Shenzhen (CN)

(72) Inventor: Hua Xie, Shenzhen (CN)

(21) Appl. No.: 19/186,552

(22) Filed: Apr. 22, 2025

(51) Int. Cl. A61H 3/04; A61B 5/00; A61B 5/1455

(52) U.S. Cl. CPC A61H 3/04; A61B 5/14552; A61B 5/6826; A61H 2201/0107; A61H 2201/5043; A61H 2201/5058; A61H 2201/5097

(57) ABSTRACT

The present application relates to a self-supporting universal monitoring assembly for a rollator, comprising: a main monitoring device detachably mounted to the rollator for interaction with a user of the rollator; a secondary sensing device detachably mounted on the rollator and configured to measure the rollator's motion parameters; the monitoring device comprising: a main body including a casing, a display screen and a control key pad; a rechargeable battery; a Main Control Board inside the casing; and a finger sleeve; a ball joint base detachably mounted on the rollator; a mounting bracket, which is detachably mounted with and supports the main body; an adjustment nut, which can adjust the assembly tightness of the mounting bracket and the ball joint base; the self-supporting universal monitoring assembly is configured to be detachably mounted onto the rollator.

### TECHNICAL FIELD

The present application relates to the field of medical assistive devices, and particularly to a universal monitoring device that can be detachably installed on a rollator to monitor the user's physiological parameters and motion status in real-time, as well as a rollator incorporating such a device.

More specifically, the present application relates to a self-supporting universal monitoring assembly that can be detachably installed onto a rollator.

### BACKGROUND

With the acceleration of the aging population, the rollator has become an important assistive tool for the elderly and people with mobility impairments. Although existing rollators have played an important role in providing support and assistance for people with mobility difficulties, there are still many limitations in the existing technology that cannot meet the growing demand for intelligentization and customization. However, traditional rollators have the following shortcomings and deficiencies:
i) Lack of versatility and adaptability: Monitoring devices are mostly designed as a plurality of components of a rollator with specific configuration and mounting design adapted to respective rollators, lacking universality and adaptability. The flexibility of installation and removal is limited, and they cannot be conveniently installed and adapted to different models of rollators. They also cannot be used, carried, or transported as an independent device separate from the rollator;
ii) Trade-off between expandability and portability: Monitoring devices, as a specific part of the rollator, need to rely on the rollator to function, lacking portability and expandability. They cannot expand functional modules according to user needs, which in turn limits the universality and expandability of the rollator. Rollator users often need to purchase additional devices when facing different scenarios and needs, increasing the cost and complexity of use;
iii) Insufficient safety monitoring: Relying only on physical structural protection, lacking real-time monitoring of the user's physiological state (such as heart rate, blood oxygen) and motion posture, unable to timely warn of fall risks, and lacking intelligent monitoring of the user's health status and motion safety; and
iv) Poor data collection, insufficient interactivity and security: Existing devices mostly only record motion distance or time, and cannot comprehensively analyze and monitor the state of the rollator user (such as gait imbalance and other abnormal states) and issue real-time warnings, posing safety hazards. This to some extent affects the confidence and sense of security of rollator users and limits the application of rollators in a wider range of scenarios.

In summary, the limitations of existing rollators mainly lie in universality, expandability, functional limitations, insufficient flexibility in disassembly and assembly, as well as defects in interactivity and security. These limitations not only affect the user experience but also restrict the further development and application of rollator technology. Therefore, there is an urgent need in this field for a self-supporting universal monitoring assembly that can be conveniently and detachably installed on a rollator, can work independently, can integrate multi-source monitoring, can be expanded with modules, and supports intelligent interaction, to meet the market demand for universal intelligent medical assistive devices and their wider application scenarios and functions, and to overcome the above-mentioned technical defects and other technical problems in the existing technology.

The information included in this section of the specification of the present application, including any references referenced herein and any descriptions or discussions thereof, are incorporated only for the purpose of reference, thus shall not to be considered as limitation to the scope of the present application.

### SUMMARY

In view of the above and other more aspects, the present application is proposed. The present application aims to address the above-mentioned deficiencies and other technical problems.

According to a main concept of the present application, the self-supporting universal monitoring assembly of the present application, which can work independently and can be manufactured, applied and sold separately, may include a main monitoring device and at least one secondary sensing device, which can work in coordination through wireless communication to achieve comprehensive data collection, processing, and early warning of physiological parameters and motion data.

More specifically, according to one aspect of the present application, there is provided a self-supporting universal monitoring assembly for a rollator, comprising: a main monitoring device, which is configured to be detachably mounted on the rollator at a position for interaction with a user of the rollator, for monitoring at least one physiological parameter of the user selected from blood oxygen level, pulse rate and body temperature; and at least one secondary sensing device in communication with the main monitoring device, which is configured to be detachably mounted on the rollator and configured to measure at least one selected from a group consisting of rollator motion time, rollator motion speed, rollator motion acceleration, rollator motion distance, rollator posture abnormality, and rollator user gait abnormality; wherein the main monitoring device comprises: a main body, which includes: a casing with a display screen and a control key pad; a rechargeable battery arranged inside the casing, for powering the main monitoring device; a Main Control Board inside the casing; and a finger sleeve located outside the main body and in communication therewith; a ball joint base, which is configured to be detachably mounted on the rollator; a mounting bracket, which is assembled with the ball joint base in a detachable manner to support the main body; and an adjustment nut, which is assembled with the ball joint base and the mounting bracket, and is configured to adjust the assembly tightness of the mounting bracket and the ball joint base, thereby facilitating adjustment and fixation of the ball joint base and the main monitoring device mounted thereon; wherein the self-supporting universal monitoring assembly is configured to be detachably mounted to the rollator.

According to one or more embodiments of the present application, the finger sleeve includes a sleeve hole for inserting the user's finger, and is equipped with a pulse oximeter for monitoring at least one of the user's blood oxygen and pulse rate, wherein the finger sleeve is in wired (e.g., via a data cable) or wireless connection with the main monitoring device; the main monitoring device is further configured with at least one of a temperature sensor, a humidity sensor, and a positioning module, wherein the positioning module is selected from a group consisting of a GPS positioning module, a Beidou positioning module, and a type of wireless tracking tag device (e.g., an AIR TAG device).

According to one or more embodiments of the present application, the main body of the main monitoring device further includes a transparent lampshade installed facing the rollator's forward direction, and an LED circuit board with an LED light installed inside the casing, wherein the LED light is configured to emit light through the transparent lampshade.

According to one or more embodiments of the present application, the main monitoring device is a separate, self-supporting device that can work independently.

According to one or more embodiments of the present application, the self-supporting universal monitoring assembly is configured as an independently operable assembly.

According to one or more embodiments of the present application, the main monitoring device is in wireless communication with the secondary sensing device.

According to one or more embodiments of the present application, the Main Control Board includes: a data processing module, configured to receive and process multi-source sensing data from the self-supporting universal monitoring assembly; a wireless communication unit, supporting one-way or two-way communication between the main monitoring device and an external medical platform; and a control circuit, configured to control the operation of the self-supporting universal monitoring assembly.

According to one or more embodiments of the present application, at least one side of the casing of the main body of the main monitoring device is provided with a T-shaped key or a T-slot for slide-in fit of an additional sensing or monitoring device.

According to one or more embodiments of the present application, the ball joint base includes: a concave arch portion, which is configured to be detachably mounted onto a handle of the rollator; a base ball head assembled on or integrally formed with the concave arch portion, with a ball head top opening at the top of the base ball head; and a ball joint inner rod assembled from below the concave arch portion, with a square inner rod, and an inner-rod through hole in the square inner rod; wherein the ball joint inner rod is assembled such that its spherical shape matches the spherical shape of the base ball head, and its square inner rod extends upward through the ball head top opening.

According to one or more embodiments of the present application, a hook is installed or formed on a side of the ball joint base.

According to one or more embodiments of the present application, the self-supporting universal monitoring assembly further includes a base fixing strap with at least one hook hole, one end of which is fixed to one side of the ball joint base, and the other end of which is engaged with the hook via the hook hole, thereby detachably fixing the ball joint base to the rollator.

According to one or more embodiments of the present application, the mounting bracket includes: a bracket socket, which is configured to match the shape of the ball joint base below, with an socket seat at the top of the bracket socket, and a socket seat hole in the socket seat matching with the square inner rod extending through the ball head top opening; two upright columns, respectively extending upward from both sides of the bracket socket; and a mounting seat supported on the two upright columns, for installing the main body of the main monitoring device thereon.

According to one or more embodiments of the present application, the height of the upright columns is set such that an installation space is formed between the bracket socket and the mounting seat.

According to one or more embodiments of the present application, the bracket socket, the upright columns, and the mounting seat are integrally formed.

According to one or more embodiments of the present application, the mounting seat is provided with a platform with two side ribs configured for detachable snap-fit of the main body of the main monitoring device.

According to one or more embodiments of the present application, the mounting seat is further provided with a wedge for snap-fit engagement.

According to one or more embodiments of the present application, the height of the installation space is sufficient to accommodate the adjustment nut.

According to one or more embodiments of the present application, the main monitoring device further includes a mounting bolt, which sequentially extends upward through the ball joint base and the socket seat hole at the top of the bracket socket, and is threadedly engaged with the adjustment nut, such that the mounting bracket can swivel relative to the ball joint base with adjustable tightness.

According to one or more embodiments of the present application, the secondary sensing device includes at least one selected from a group consisting of a Hall sensor, a GPS positioning module, a contact position sensor, a proximity sensor, a gyroscope, a magnetic rotary encoder, a strain gauge, an accelerometer, a haptic feedback sensor, a LIDAR sensor, and any combination thereof.

According to one or more embodiments of the present application, the main monitoring device further includes an alarm module, which is configured to be triggered manually or automatically to issue at least one of an audible alarm, a light alarm, and an SOS distress signal, for example, when an early warning signal exceeding the set threshold is analyzed or received, or when the user detects or feels the need to raise an alarm for help.

According to another aspect of the present application, there is provided a rollator, comprising the self-supporting universal monitoring assembly as described above detachably mounted thereon.

According to one or more embodiments of the present application, the rollator is electric or manual.

According to one or more embodiments of the present application, the main monitoring device may include: a three-dimensional motion modeling unit, which constructs a user gait dynamics model based on the wheel-axis acceleration data and handle-tilt angle data collected from the secondary sensing device; and an abnormality detection engine, which uses a convolutional neural network optimized by transfer learning to identify abnormal gait patterns in real-time.

According to one or more embodiments of the present application, the self-supporting universal monitoring assembly is configured to monitor the user's exercise intensity index. By collecting the user's pulse rate and age, the exercise intensity index can be calculated and evaluated to determine whether it is at a high, moderate, or low level.

According to one or more embodiments of the present application, the self-supporting universal monitoring assembly is configured to monitor the user's energy expenditure during each use of the rollator, i.e., calories burned. For example, the calorie value can be calculated using the following formula:

Calories (kcal)=MET×Weight (kg)×Time (hours),
wherein Weight represents the weight of the user of the rollator, Time represents the walking time of the user during each use of the rollator, and the MET (Metabolic Equivalent) parameter is set as follows:
   Slow walking (3-4 km/h): MET≈3.0;
   Normal walking (5 km/h): MET≈4.3;
   Fast walking (6.5 km/h): MET≈5.0; and
   Typically, assuming the user moves with the rollator at a normal walking speed (5 km/h), the MET is set to 4.3.

The present application can also achieve other advantageous technical effects that are not listed one by one, some of which may be partially described in the following text, and are apparent to those skilled in the art after reading the present application.

### BRIEF DESCRIPTION OF THE DRAWINGS

The above features and advantages and other features and advantages of these embodiments, and the implementing way thereof, would be apparent to those skilled in the art, and the present application may be better understood, by reference to the following Figures and the related description.

FIG. 1 is a schematic perspective view of a main monitoring device according to an embodiment of the present application.

FIG. 2 is a schematic perspective view of the main monitoring device shown in FIG. 1 from another perspective, particularly showing the window of the main monitoring device in this embodiment.

FIG. 3 is a schematic perspective view of an example of the secondary sensing device according to an embodiment of the present application, particularly showing the Hall sensor and the magnet that works in coordination with it.

FIG. 4 is a schematic perspective view of a rollator detachably installed with the main monitoring device and secondary sensing device shown in FIGS. 1-3, particularly showing the rollator from the perspective of a user using the rollator viewing forward.

FIG. 5 is a schematic perspective view of the rollator shown in FIG. 4 from the side.

FIG. 6 is a schematic perspective view of the base structure of the main monitoring device according to an embodiment of the present application.

FIG. 7 is an exploded view of the base structure shown in FIG. 6.

FIG. 8 is an exploded view of the main body of the main monitoring device according to an embodiment of the present application.

FIG. 9 is a schematic perspective view of a main monitoring device according to another example, which differs from the main monitoring device shown in FIG. 1 in that an S button is added to the control key pad.

FIG. 10 is a schematic perspective view of the main monitoring device shown in FIG. 9 from another perspective, particularly showing the window of the main monitoring device of FIG. 9.

The reference numerals and the corresponding features is listed as follows: 100 Main monitoring device; 110 Main body; 1101 Casing; 1101A upper casing; 1101B Lower casing; 1102 Display screen; 1102A Protective transparent diaphragm; 1103 Control key pad; 1103A S button; 1104 T-shaped key; 1105 Transparent lampshade; 1106 Main Control Board; 1107 LED circuit board; 1108 Rechargeable battery; 1109 Waterproof plug; 1109A USB port; 120 Ball joint base; 1201 Concave arch portion; 1202 Hook; 1203 Base ball head; 1203A ball head top opening; 1204 Ball joint inner rod; 1204A Square inner rod; 1204B Inner-rod through hole; 1205 Mounting bolt; 1205A Hexagonal bolt head; 130 Mounting bracket; 1301 Bracket socket; 1301A Socket seat; 1301B Socket seat hole; 1302 upright column; 1303 Grip portion; 1304 Mounting seat; 1304A Side rib; 1304B Side rib; 1304C Wedge; 1304D wedge; 1304E platform; 140 Data cable; 150 Finger sleeve; 1501 Sleeve hole; 160 Base fixing strap; 1601 Hook hole; 1602 Arch adapter; 170 Adjustment nut; 200 Secondary sensing device; 210 Hall sensor; 220 Fixing strap; 2101 Hall sensor head; 230 Tie band; 240 Magnet; 300 Rollator; 310 Wheel; 320 Main frame; 330A Right handle; 330B Left handle.

### DETAILED DESCRIPTION

Details of one or more embodiments of the present application will be set forth in the following description of the accompanying drawings. Other features, objects, and advantages of the present application may be apparent from these descriptions, drawings, and claims.

It should be understood that the illustrated and described embodiments are not limited to the details of configuration and arrangement of components set forth in the following description or illustrated in the accompanying drawings. The examples are provided by way of explanation of the disclosed embodiments and not by way of limitation. Indeed, it will be apparent to those skilled in the art that various modifications and variations can be made to the embodiments of the present application without departing from the scope or spirit of the present application.

The present application will be described in more detail below with reference to the attached drawings and the embodiment(s).

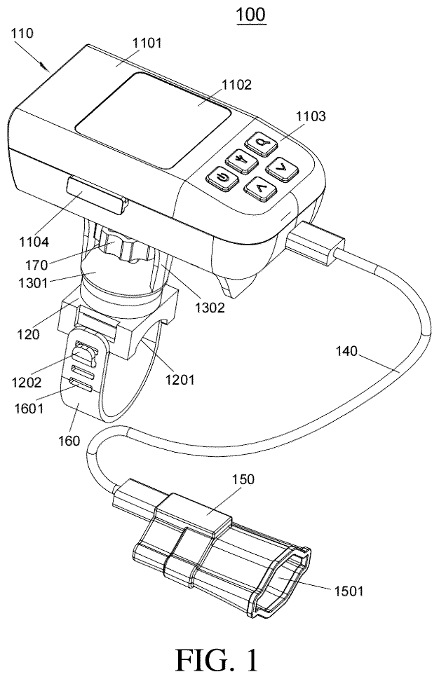

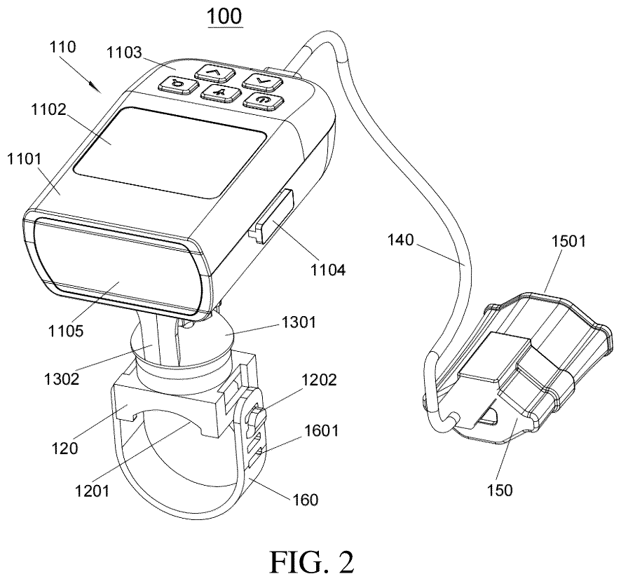

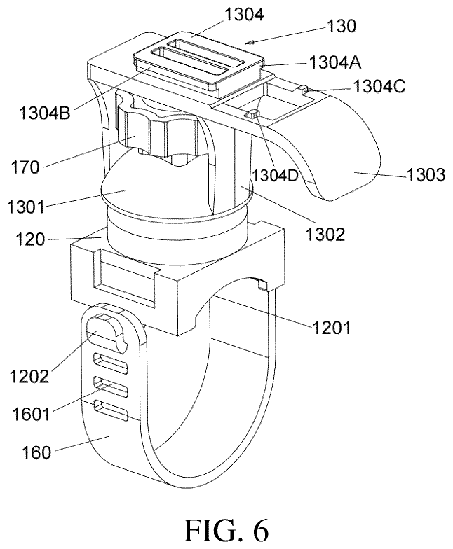

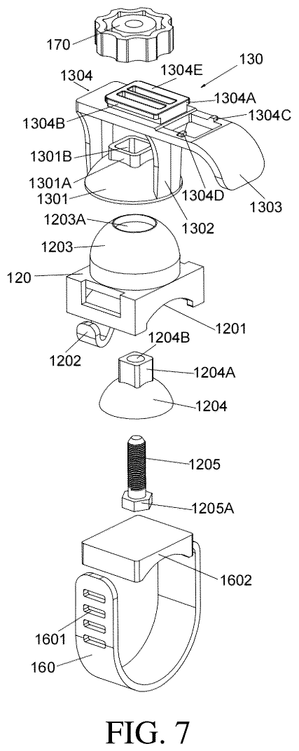

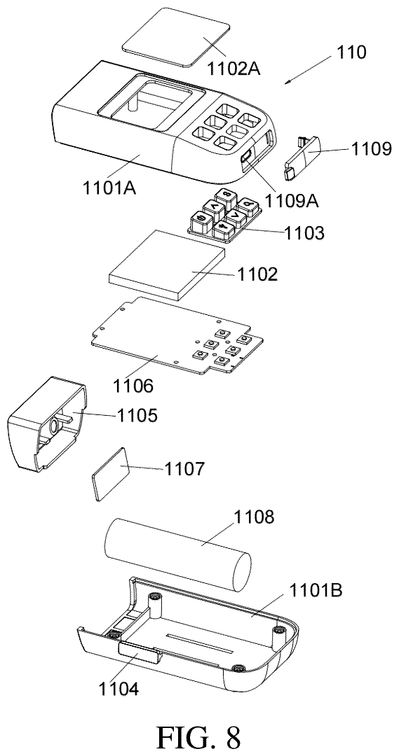

FIG. 1 is a schematic perspective view of a main monitoring device 100 according to an embodiment of the present application. FIG. 2 is a schematic perspective view of the main monitoring device 100 shown in FIG. 1 from another perspective, particularly showing the window of the main monitoring device 100 in this embodiment. FIG. 8 is a schematic exploded view of the main body of the main monitoring device 100 according to an embodiment of the present application. FIG. 6 is a schematic perspective view of the base structure of the main monitoring device 100 According to one or more embodiments of the present application, and FIG. 7 is a schematic exploded view of the base structure shown in FIG. 6.

According to one or more embodiments of the present application, as shown in FIGS. 1-8, a self-supporting universal monitoring assembly for a rollator is disclosed, which is a separate and independently functioning product which can be self-supported and self-standing. This type of product may be manufactured, commercialized, and utilized as an independent, self-contained entity, while maintaining autonomous operation and fulfilling its intended operational objectives without reliance on external systems, and may further interface with rollators or external systems to achieve enhanced customization capabilities. Functionality. In addition, it has good adaptability, adaptability, and flexibility in configuration and installation methods, so it can be easily installed on various different types of rollators for work, and can also be easily removed for updating, maintenance, replacement, and upgrade, thus being suitable for both the original equipment market and the aftermarket, with very good universality and a wide range of application scenarios.

As shown in the figures, the self-supporting universal monitoring assembly for a rollator in this embodiment may include: a main monitoring device 100, which is detachably mounted on the rollator at a location convenient for use and interaction with the rollator user, for monitoring various physiological parameters and indicators of the rollator user, etc.

As shown in FIGS. 1-2, 6-8, the main monitoring device 100 may include, for example, an integral main body of the main monitoring device 110. Preferably, the main monitoring device 100 is an independent self-supporting device that can work independently.

The main body of the main monitoring device 110 may include a casing 1101, which may include an upper casing 1101A and a lower casing 1101B that are snapped together, as shown in FIG. 8. On the side of the casing 1101, for example, on one or both sides of the lower casing 1101B, there may be provided a sliding-in mortise and tenon interface for supporting expansion modules, such as T-shaped key 1104, or a T-slot (not shown), for slide-in fit of an additional sensing or monitoring device, including but not limited to temperature sensors, etc. If the installation part on the casing 1101 is a T-shaped key 1104, then the additional sensing or monitoring device will have a cooperating T-slot (not shown), and vice versa. The advantage of this key-slot fit construction is that it can achieve quick and reliable assembly and disassembly. As an example, the casing 1101 may be made of lightweight ABS material, and may contain a rechargeable battery, a Main Control Board, an LED circuit board, a control circuit, a communication and signal processing unit/circuit, etc.

On the casing 1101 (for example, as shown in the figure, on the upper casing 1101A), there may be provided a display screen 1102 and a control key pad 1103. The display screen 1102, which may be covered by a protective transparent diaphragm 1102A, can display the user's physiological parameters and the rollator's motion status in real-time, and is configured to display various information for the rollator user, including local information, such as the measured physiological parameters of the user (e.g., blood oxygen, pulse rate, speed, posture), possible alarm information, and optional environmental data information (e.g., environmental temperature, environmental humidity), as well as possible remote information, such as information or warnings from remote sources (e.g., from a doctor or guardian), etc, through arrangement of sensors/transducers in the main monitoring device 100 and/or the secondary sensing device 200, such as pulse oximeter, accelerator, gyro, velocity transducer, accelerator, temperature sensor, humidity sensor, and so on. The control key pad 1103 allows the user to perform simple operations, such as starting or stopping monitoring, setting alarm thresholds, turning the power on/off, and turning the LED light on/off, etc. The intuitive human-machine interaction interface of the display screen 1102 and the control key area 1103 makes it easy for users to get started without complicated training.

Inside the casing 1101, there is provided a rechargeable battery 1108 for powering the self-supporting universal monitoring assembly and the main monitoring device 100. The built-in rechargeable battery 1108 provides independent power supply capability for the entire main monitoring device (and part of the secondary sensing device 200). This design makes the main monitoring device self-supporting, independent of external power sources, and capable of working independently in various environments, greatly enhancing the flexibility and portability of the device.

Inside the casing 1101, there is also provided a Main Control Board 1106, i.e., a PCB mainboard for data storage, processing, and data communication, including local communication, communication with the secondary sensing device 200, and communication with remote platforms or servers, etc.

A finger sleeve 150 may be provided outside the main body of the main monitoring device 110 and in communication connection therewith, for monitoring the user's physiological characteristic parameters such as pulse rate and blood oxygen. The finger sleeve 150 may be constructed with a sleeve hole 1501 for insertion of the user's finger, and is equipped with a pulse oximeter for monitoring at least one of the user's blood oxygen and pulse rate, wherein the finger sleeve 150 may be connected to the main monitoring device 100 via a data cable 140 such as with a type C USB connector to connect with a USB port 1109A, to achieve physiological monitoring. The USB port 1109A, if not in use, will be covered with a waterproof plug 1109 to avoid water or dust invasion.

The main monitoring device 100 may include a ball joint base 120, and the main monitoring device 100 can be installed onto the ball joint base 120 via a mounting bracket 130 (as described in detail below) in a swivelable way. The ball joint base 120 is constructed to be conveniently detachably mounted on the rollator, thereby enabling the main monitoring device 100 to be very conveniently and detachably mounted on the rollator, for example, on the handle 330A or 330B, and can be rotated relative to the handle.

Assembled together with the ball joint base 120 is the mounting bracket 130, which supports and detachably mounts the main body of the main monitoring device 110. Thus, when the ball joint base 120 is detachably mounted onto the rollator, the main monitoring device 100 can also be very conveniently detachably mounted onto the rollator, as further described below.

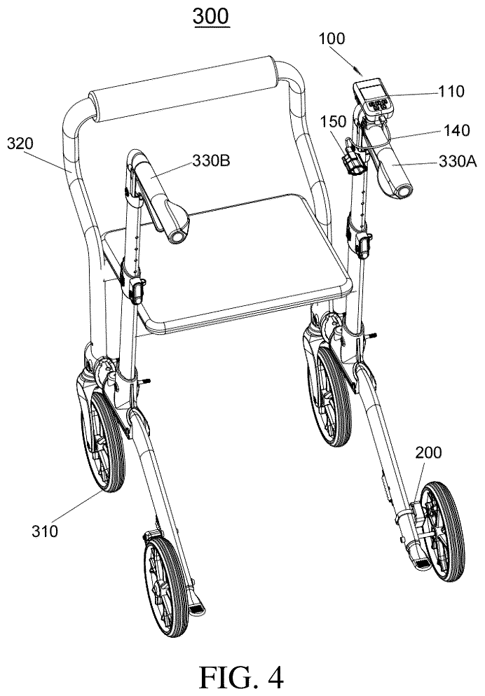

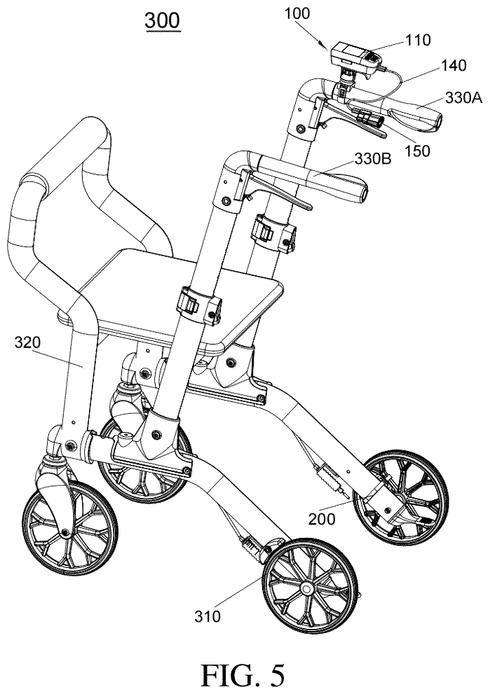

The ball joint base 120 includes a concave arch portion 1201, which is configured to be conveniently mounted and fixed on the rollator's handle, which is generally rod-shaped. Generally speaking, for example, the arch/arc shape and size of the concave arch portion 1201 match the generally cylindrical handle of the rollator (e.g., the handle 330A shown in FIGS. 4-5).

The base ball head 1203 is provided (e.g., assembled or integrally formed) on the concave arch portion 1201, with a ball head top opening 1203A at the top of the base ball head 1203. The ball joint inner rod 1204 is installed (e.g., integrally formed or fixedly assembled) below the concave arch portion 1201, with a square inner rod 1204A at the top, and an inner-rod through hole 1204B at the top of the inner rod 1204. The shape and size of the ball joint inner rod 1204 are constructed such that the lower part of its ball joint (e.g., as shown in FIG. 7) matches the spherical shape of the base ball head 1203, and its upper part of the square inner rod 1204A extends upward through the ball head top opening 1203A.

The mounting bracket 130 includes a bracket socket 1301, which is configured to match the shape of the ball joint base 120 below. An socket seat 1301A is provided at the top of the bracket socket 1301, and the socket seat hole 1301B in the socket seat 1301A is constructed to match with the square inner rod 1204A extending through the ball head top opening 1203A. Preferably, two upright columns 1302, which are generally diametrically opposite, extend upward from both sides of the bracket socket 1301, and are fixedly connected (preferably, integrally formed) with the mounting seat 1304 above. The mounting seat 1304 is supported on top of the two upright columns 1302, and is used for installing the main monitoring device 100 (the main body of the main monitoring device 110).

There is a sufficiently large gap between the bracket socket 1301 and the mounting seat 1304 of the mounting bracket 130 to form an installation space for the adjustment nut 170. Specifically, the height of the upright columns 1302 is set such that a certain height of installation space is formed between the bracket socket 1301 and the mounting seat 1304. The height of the installation space is sufficient to accommodate the adjustment nut 170, as shown in FIGS. 6-7, where for clarity of illustration, the position of the adjustment nut 170 in the exploded view 7 is shown above the mounting bracket 130, but in fact, after assembly, it is located in the installation space between the mounting bracket 130 and the ball joint base 120.

Preferably, the bracket socket 1301, upright columns 1302, and mounting seat 1304, for example, are integrally formed, such as an injection-molded plastic part, such that the mounting bracket 130 is an integral part.

The mounting seat 1304 is provided with a platform 1304E with two side ribs 1304A and 1304B, which facilitates the removable slide-in fit of the main body of the main monitoring device 110 (which has grooves, not shown, to cooperate with the ribs 1304A and 1304B). The grip portion 1303 can be gripped by the user's finger facilitating for installation, removal and orientation adjustment. The ribs 1304A and 1304B are constructed together with the corresponding side surfaces of the platform 1304E to form an undercut structure, allowing the main body of the main monitoring device 110 to be slid in and out along the direction of extension of the ribs 1304A and 1304B, while being reliably fixed and positioned in other directions. The mounting seat 1304 may further be provided with wedges 1304C and 1304D for snap-fit engagement to securely fix the main body of the main monitoring device 110. After the main body of the main monitoring device 110 is slid in along the ribs 1304A and 1304B, it can be further fixed on the mounting seat 1304 by snap-fit engagement, ensuring that once it is slid in and installed, it is also restrained in place along the direction of extension of the ribs 1304A and 1304B, thereby ensuring the all-around fixation of the main monitoring device 100 after installation.

The main monitoring device 100 also includes a mounting bolt 1205 configured together with the above-mentioned structure. The mounting bolt 1205, for example, as shown in FIG. 7, extends upward through the ball joint base 120 and the socket seat hole 1301B at the top of the bracket socket 1301, and is threadedly engaged with the adjustment nut 170. This allows the mounting bracket 130 to be rotatably positioned, adjusted and fixed relative to the ball joint base 120 with an adjustable tightness within the horizontal plane by 360 degrees, such as by loosening and adjusting the adjustment nut 170. As an example, the square inner rod 1204A extend upwardly through the socket seat hole 1301B in the socket seat 1301A of the bracket socket 1301 and held in place by mounting bolt 1205, and the sizes of the mounting bolt 1205 and the square inner rod 1204A can be set smaller than the size of the socket seat hole 1301B and the ball head top opening 1203A. Due to the adjustable characteristics of this ball-socket joint, the mounting bracket 130 can be tilted to a certain extent relative to the ball joint base 120 (e.g., a tilt angle of ±20°) and can be tilted to the left and right (e.g., a tilt angle of ±15°), which is understandable to those skilled in the art. After adjusting to the desired position, it can be fixed in place by tightening the adjustment nut 170.

The self-supporting universal monitoring assembly also includes a base fixing strap 160 with at least one hook hole 1601 that is provided for engagement with a hook 1202 provided (e.g., integrally formed) on one side of the ball joint base 120, as shown in FIGS. 6-7. One end of the base fixing strap 160 is provided with an arch adapter 1602 which is installed and secured to the ball joint base 120, and the other end with hook holes 1601, is engaged with the hook 1202, thereby detachably fixing the ball joint base 120 to the rollator. As an example, the ball joint base 120 with hook 1202 is an integral injection-molded plastic part, and the base fixing strap 160 is an injection-molded part made of silicone elastomer (such as Polydimethylsiloxane), with an integrally formed arch adapter 1602 at one end of the base fixing strap 160 being inserted in the concave arch portion 1201 from the other side of the ball joint base 120 opposite to the hook 1202, and fixed in the concave arch portion 1201 such as by snap-fit or interference fit.

Of course, those skilled in the art will understand that other designs for detachably installing the self-supporting universal monitoring assembly via the ball joint base 120 onto the rollator (e.g., the handle of the rollator) are also feasible, such as Velcro, adjustable fixing rings, etc., all of which are within the scope of the present application.

Further described in conjunction with the preceding text, a mounting bolt 1205 with, for example, a hexagonal bolt head 1205A can be extended upward through the ball joint base 120 and the mounting bracket 130, and an adjustment nut 170 can be screwed into the installation space position of the mounting bracket 130, thereby integrating the entire installation structure, as shown in FIGS. 6-7. More specifically, the installation position of the adjustment nut 170 is configured to adjust the tightness of the assembly of the mounting bracket 130 and the ball joint base 120, thereby facilitating the adjustment and fixation of the ball joint base 120 and the monitoring device installed thereon in the rollator; wherein the self-supporting universal monitoring assembly is configured to be detachably mounted to the rollator.

As an example, the main body of the main monitoring device 110 may further include a transparent lampshade 1105 installed facing the rollator's forward direction as a window, and an LED circuit board 1107 with an LED light installed inside the casing 1101. The light from the LED light (not shown) of the LED circuit board 1107 can pass through the transparent lampshade 1105 and be emitted, as shown in FIG. 2, for auxiliary illumination. Of course, those skilled in the art will understand that the LED light can not only be used for auxiliary illumination, but also additionally for light warning on the rollator. Due to the flexible adjustability of the installation structure, the direction of illumination and warning of the LED light is not limited to the forward direction shown in FIGS. 1-2, but can be oriented in any desired direction.

In addition, as an example, the Main Control Board 1106 may also include a data processing module (not shown), which is configured to receive and process multi-source sensing data from the self-supporting universal monitoring assembly. The Main Control Board 1106 may also include a wireless communication unit (not shown) to support one-way or two-way communication with the secondary sensing device 200, and if required, further with an external medical platform (not shown).

The Main Control Board 1106 also includes a control circuit, which is configured to control the operation of the self-supporting universal monitoring assembly, as well as to support interaction with the user, for example, through the control key pad 1103.

As an example of intelligent early warning, the Main Control Board 1106 may integrate an alarm module that triggers an on-site audiovisual alarm and/or sends an SOS signal to the patient's family and/or medical platform when physiological parameters are abnormal (e.g., blood oxygen below the threshold) or motion risks (e.g., sudden acceleration) are detected.

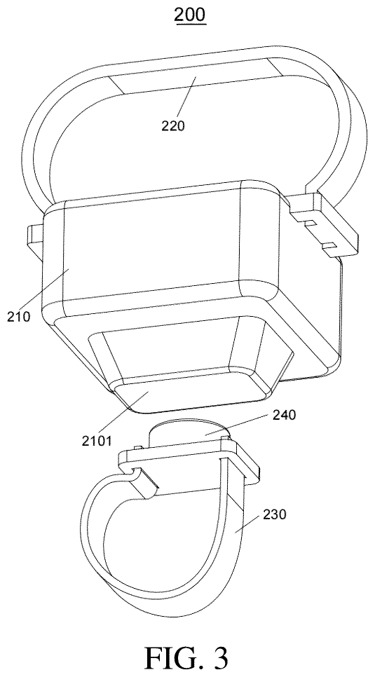

As shown in FIGS. 1-3, the self-supporting universal monitoring assembly for a rollator in this embodiment may also optionally include at least one secondary sensing device 200 in communication with the main monitoring device 100, to achieve more configurations, functions, and uses. The secondary sensing device 200 is detachably mounted onto the rollator 300 and is configured to measure at least one of the rollator's motion speed, motion acceleration, motion distance, rollator posture abnormality, and rollator user gait abnormality. FIG. 3 is a schematic perspective view of an example of the secondary sensing device 200 According to one or more embodiments of the present application, particularly showing the Hall sensor 210 and the magnet 240 that works in cooperation with it. For example, as shown in FIGS. 1-3, the magnet 240 is fixed to the rotatable wheel 310 of the rollator with a tie band 230, and the Hall sensor head 2101 of the Hall sensor 210 is fixed to the axle mounting bracket with a fixing strap 220, located in a neighboring position relative to the magnet 240, for sensing the magnetic field of the magnet 240.

Of course, those skilled in the art will understand that in addition to the Hall sensor 210, the secondary sensing device 200 may also include other forms of sensors or detection devices, such as, for example, a GPS positioning module, a contact position sensor, a proximity sensor, a gyroscope, a magnetic rotary encoder, a strain gauge, an accelerometer, haptic feedback sensor, a LIDAR sensor, or any combination thereof. For example, depending on the application scenario and functional requirements, the secondary sensing device 200 may also selectively include at least one of the following sensors (arrays):

Motion sensing configuration example: 6-axis IMU (MPU6050) measures attitude angles (roll/pitch/yaw), acceleration (±16 g), and gyroscope data.

Environmental sensing configuration example: ToF LIDAR (ranging from 0.1-10 m, accuracy±2 cm) builds a 3D map around the rollator and detects obstacle distances.

Mechanical sensing configuration example: Strain-type pressure sensor (FSR 4060) installed at the wheel axle center monitors changes in ground friction.

To achieve long battery life, the secondary sensing device 200, also provided with a battery, such as button battery, may also be designed for low power consumption, with dynamic power management enabling a battery life of up to 120 hours, where the current in idle mode is configured to be≤0.5 mA, and the current in active mode is configured to be≤50 mA. For the secondary sensing device 200, other battery with longer battery life, or alternatively a wired power supply from such as the main monitoring device 110, may be anticipated.

FIG. 4 is a schematic perspective view of the rollator 300 detachably installed with the main monitoring device 100 and secondary sensing device 200 shown in FIGS. 1-3, particularly showing the rollator 300 from the perspective of a user using the rollator viewing forward. FIG. 5 is a schematic perspective view of the rollator shown in FIG. 4 from the side. As shown in FIGS. 4-5, both the main monitoring device 100 and the secondary sensing device 200 are installed onto the rollator 300, where the main monitoring device 100 is detachably mounted on the right handle 330A (of course, it can also be mounted onto the left handle 330B), and the secondary sensing device 200 is installed on one of the wheels 310. The main monitoring device 100 and the secondary sensing device 200 communicate wirelessly, for example, via Bluetooth or Wi-Fi.

Those skilled in the art will understand that the rollator 300 shown in the figures above can be electric or manual.

As an example, the Main Control Board 1106 may also specifically include an alarm module or alarm circuit, which is configured to issue an audible alarm, a light alarm, or an SOS distress signal, when an early warning signal exceeding the set threshold is analyzed or received.

To provide the rollator and the self-supporting universal monitoring assembly installed thereon with user fall detection and alarm functions, the main monitoring device 100 of the self-supporting universal monitoring assembly may be configured to include: a three-dimensional motion modeling unit, which constructs a user gait dynamics model based on the wheel-axis acceleration data and handle-tilt angle data collected from a secondary sensing device 200; and an abnormality detection engine, which uses a convolutional neural network optimized by transfer learning to identify abnormal gait patterns in real-time. When an abnormal gait pattern appears, that is, when an abnormal situation such as a user fall or a rollator out-of-control occurs, an on-site or remote alarm signal can be sent out.

### A Variant Example of the Main Monitoring Device 100

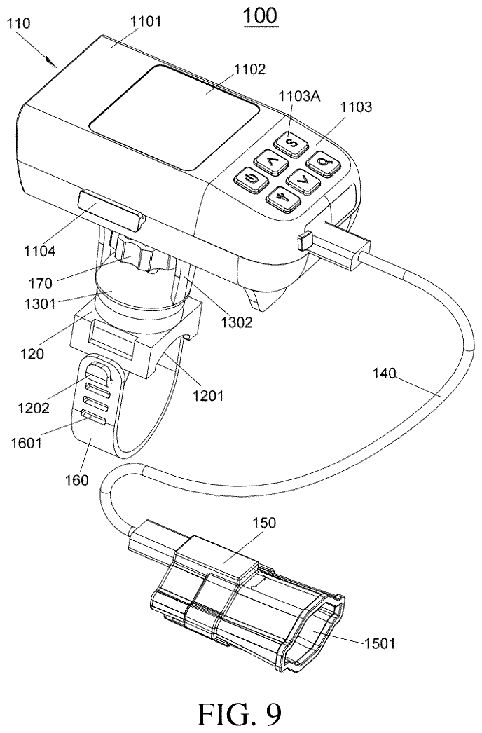

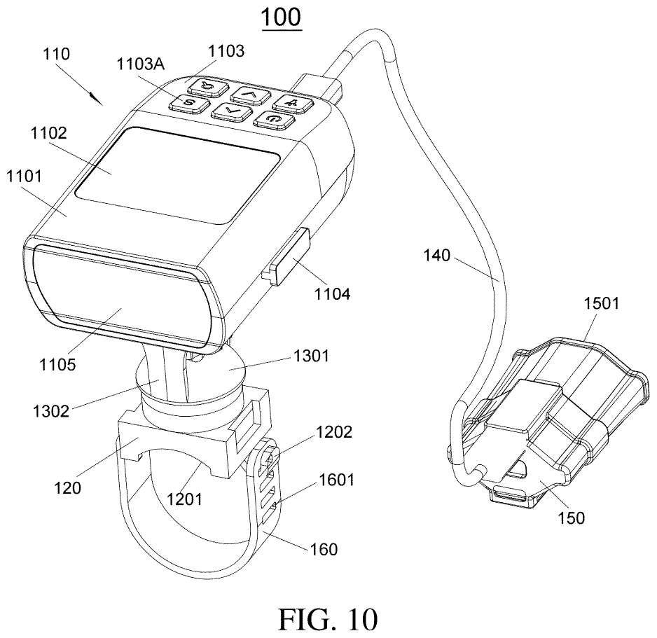

FIG. 9 is a schematic perspective view of the main monitoring device 100 according to a variant example, which is substantially the same as the main monitoring device 100 shown in FIG. 1, with a difference that an S button 1103A being added in the control key pad 1103. FIG. 10 is a schematic perspective view of the main monitoring device shown in FIG. 9 from another perspective, particularly showing the window of the main monitoring device 100 of FIG. 9.

In the main monitoring device shown in FIGS. 9-10, in addition to the configuration (e.g., temperature sensor, pulse oximeter, etc.) of the main monitoring device 100 according to the embodiment of FIG. 1, at least one of a positioning module, a humidity sensor, and an alarm device may be further integrated therewith.

For example, the positioning module may be a GPS positioning module, a Beidou positioning module, or a wireless tracking tag device like an AIR TAG device, etc. Among them, the AIR TAG device is a preferred one, which can be conveniently attached to the main monitoring device 100 or also to other parts suitable for interaction with the self-supporting universal monitoring assembly. The AIR TAG device is not only easy to install, but also can be conveniently located and tracked through the user's or guardian's Apple mobile device, and can alternatively or additionally communicate with the self-supporting universal monitoring assembly (the main monitoring device or the secondary monitoring device thereof).

An example of the alarm device may be an SOS alarm device, which can be provided in the main monitoring device 100 (e.g., integrated into the main monitoring device 100), and can be triggered by manually pressing the associated S button 1103A, to produce an on-site audiovisual alarm, for example, by the speaker/LED light of the main monitoring device 100 to emit a flashing/buzzing alarm representing an SOS distress signal. Additionally or alternatively, the SOS alarm device may also be automatically triggered to send a remote alarm, transmitting preset distress information (such as location, contact information) to emergency contacts, guardians, or rescue agencies, etc.

Below is a further description of the installation, adjustment, and operation of the self-supporting universal monitoring assembly according to this embodiment of the present application in conjunction with the drawings and the above-mentioned content.

### Installation and Adjustment of the Main Monitoring Device 100

Installation of the ball joint base 120: The concave arch portion 1201 is installed onto the rollator handle 330A and reinforced by winding the base fixing strap 160, with the hook 1201 hooked into the appropriate hook hole 1601 to be securely fastened in place.

Bracket angle adjustment: Loosen the adjustment nut 170, swivel the mounting bracket 130 to the desired viewing angle and orientation, and then tighten the adjustment nut 170 to ensure that the display screen 1102 and control key pad 1103 of the main monitoring device 100 are properly oriented for easy viewing and interaction by the user.

Installation of the main monitoring device 100: Slide the main body of the main monitoring device 110 along the ribs 1304A and 1304B of the platform 1304E, and lock it in place with the wedge 1304C.

### Example of Operation of the Self-Supporting Universal Monitoring Assembly

Data acquisition: The finger sleeve 150 continuously collects the user's blood oxygen data; the secondary sensing device 200 real-time collects various parameters of the rollator, such as motion speed, acceleration, motion distance, as well as data related to rollator posture abnormality and rollator user gait abnormality, etc., and sends these parameters to the main monitoring device 100 via wireless communication (e.g., Bluetooth).

Data processing and early warning: The data processing module of the Main Control Board 1106 analyzes abnormal gait (e.g., sudden acceleration), triggers LED light flashing and buzzer alarm, and can selectively send SOS alarm information and location information to preset contacts via the GPS module at the same time.

Medical platform connection: The wireless communication unit supports 4G/5G, Wi-Fi and/or Bluetooth communication, enabling one-way or two-way communication between the main monitoring device and an external medical platform, uploading data to the cloud in real-time for remote diagnosis by doctors.

Electronic fence function: The main monitoring device 100 may integrate a GPS module, which can implement an electronic fence function in combination with rollator motion speed data to prevent rollator users from exceeding the safe activity range.

Some of the advantages and application scenarios of the present application are as follows.

### Advantageous Technical Effects

Universality and high flexibility: The universal modular design supports quick disassembly and functional expansion, meeting different user needs and a variety of rollator types.

Comprehensive monitoring capabilities: The main and secondary devices work in coordination to simultaneously collect physiological and motion data, enhancing the accuracy of health management and safety early warning.

Enhanced safety: Blood oxygen/heart rate abnormality monitoring (with customizable thresholds), triggering a three-level alarm when blood oxygen is less than 90% or resting heart rate is greater than 120 bpm; fall prediction accuracy reaches 92% (based on 100,000 hours of clinical data training), with a response time of less than 200 ms.

Ease of use: The rollator can be adapted and installed within 30 seconds, and supports quick-release maintenance; the self-adaptive clamping mechanism is compatible with 90% of the rollator handle specifications available on the market.

Intelligent services: It is easy to expand with an AI gait analysis module to provide gait abnormality alarms and rehabilitation training suggestions (e.g., gait optimization); adjustable-brightness LED lighting assists operation in low-light environments.

Cost-effectiveness: The universal modular design of the self-supporting universal monitoring assembly of the present application greatly improves the convenience of functional/smart expansion of traditional rollators and reduces the cost of expansion.

### Application Scenarios

Home care: When the elderly use the rollator equipped with the self-supporting universal monitoring assembly according to the present application, an alarm can be automatically triggered if an abnormal state is detected. Children/guardians can remotely monitor the living situation of the elderly living alone through an APP to prevent accidents during nighttime outings.

Medical institutions: Used for patient health/vital signs monitoring, with data synchronized to the cloud/medical platform in real-time.

Rehabilitation monitoring: Postoperative rehabilitation monitoring for surgical patients, real-time transmission of vital signs to medical staff; doctors adjust rehabilitation plans based on motion data and compare the improvement of gait before and after treatment.

Outdoor travel: GPS positioning+electronic fence function to prevent rollator users from deviating from designated areas (e.g., elderly care communities).

The foregoing description of various embodiment of the present application is presented for purposes of illustration. The foregoing description is not intended to be exhaustive, nor is it intended to limit the present application to the precise features and/or forms as disclosed above. Obviously, many modifications and variations can be made in light of the above teachings. It is intended that the scope of the present application will be defined by the appended claims.

What is claimed is:

1. A self-supporting universal monitoring assembly for a rollator, comprising:
a main monitoring device, which is configured to be detachably mounted on the rollator at a position for interaction with a user of the rollator, for monitoring at least one physiological parameter of the user selected from blood oxygen level, pulse rate and body temperature; and
at least one secondary sensing device in communication with the main monitoring device, which is configured to be detachably mounted on the rollator and configured to measure at least one selected from a group consisting of rollator motion time, rollator motion speed, rollator motion acceleration, rollator motion distance, rollator posture abnormality, and rollator user gait abnormality;
wherein the main monitoring device comprises:
a main body, which includes: a casing with a display screen and a control key pad; a rechargeable battery arranged inside the casing, for powering the main monitoring device; a Main Control Board inside the casing; and a finger sleeve located outside the main body and in communication therewith;
a ball joint base, which is configured to be detachably mounted on the rollator;
a mounting bracket, which is assembled with the ball joint base in a detachable manner to support the main body; and
an adjustment nut, which is assembled with the ball joint base and the mounting bracket, and is configured to adjust the assembly tightness of the mounting bracket and the ball joint base, thereby facilitating adjustment and fixation of the ball joint base and the main monitoring device mounted thereon;
wherein the self-supporting universal monitoring assembly is configured to be detachably mounted to the rollator.

2. The self-supporting universal monitoring assembly according to claim 1, wherein the finger sleeve includes a sleeve hole for inserting the user's finger, and is equipped with a pulse oximeter for monitoring at least one of the user's blood oxygen and pulse rate, wherein the finger sleeve is in wired or wireless connection with the main monitoring device; and
wherein the main monitoring device is further provided with at least one of a temperature sensor, a humidity sensor, and a positioning module selected from a group consisting of a GPS positioning module, a Beidou positioning module, and a wireless tracking tag device.

3. The self-supporting universal monitoring assembly according to claim 1, wherein at least one side of the casing of the main body of the main monitoring device is provided with a T-shaped key or a T-slot for slide-in fit of an additional sensing or monitoring device.

4. The self-supporting universal monitoring assembly according to claim 1, wherein the Main Control Board includes:
a data processing module, configured to receive and process multi-source sensing data from the self-supporting universal monitoring assembly;
a wireless communication unit, supporting one-way or two-way communication between the main monitoring device and the secondary sensing device; and
a control circuit, configured to control the operation of the self-supporting universal monitoring assembly.

5. The self-supporting universal monitoring assembly according to claim 4, wherein the main monitoring device is in Bluetooth or Wi-Fi communication with the secondary sensing device.

6. The self-supporting universal monitoring assembly according to claim 4, wherein the wireless communication unit is configured to support one-way or two-way communication between the main monitoring device and an external medical platform.

7. The self-supporting universal monitoring assembly according to claim 1, wherein the main body of the main monitoring device further includes a transparent lampshade installed facing the rollator's forward direction, and an LED circuit board with an LED light installed inside the casing.

8. The self-supporting universal monitoring assembly according to claim 1, wherein the ball joint base includes:
a concave arch portion, which is configured to be detachably mounted onto a handle of the rollator;
a base ball head assembled onto or integrally formed with the concave arch portion, with a ball head top opening at the top of the base ball head; and
a ball joint inner rod assembled from below the concave arch portion, with a square inner rod, and an inner-rod through hole in the square inner rod;
wherein the ball joint inner rod is assembled such that its spherical shape matches the spherical shape of the base ball head, and its square inner rod extends upward through the ball head top opening.

9. The self-supporting universal monitoring assembly according to claim 8, wherein a hook is installed or formed on a side of the ball joint base.

10. The self-supporting universal monitoring assembly according to claim 9, wherein the self-supporting universal monitoring assembly further includes a base fixing strap with at least one hook hole, one end of which is fixed to one side of the ball joint base, and the other end of which is engaged with the hook via the hook hole, thereby detachably fixing the ball joint base to the rollator.

11. The self-supporting universal monitoring assembly according to claim 8, wherein the mounting bracket includes:
a bracket socket, which is configured to match the shape of the ball joint base below, with a socket seat at the top of the bracket socket, and a socket seat hole in the socket seat matching with the square inner rod extending through the ball head top opening;
two upright columns, respectively extending upward from both sides of the bracket socket; and
a mounting seat supported on the two upright columns, for installing the main body of the main monitoring device thereon.

12. The self-supporting universal monitoring assembly according to claim 11, wherein the height of the upright columns is set such that an installation space is formed between the bracket socket and the mounting seat.

13. The self-supporting universal monitoring assembly according to claim 11, wherein the bracket socket, the upright columns, and the mounting seat are integrally formed.

14. The self-supporting universal monitoring assembly according to claim 11, wherein the mounting seat is provided with a platform with two side ribs configured for detachable snap-fit of the main body of the main monitoring device.

15. The self-supporting universal monitoring assembly according to claim 14, wherein the mounting seat is further provided with a wedge for snap-fit engagement.

16. The self-supporting universal monitoring assembly according to claim 12, wherein the height of the installation space is sufficient to accommodate the adjustment nut.

17. The self-supporting universal monitoring assembly according to claim 11, wherein the main monitoring device further includes a mounting bolt, which sequentially extends upward through the ball joint base and the socket seat hole at the top of the bracket socket, and is threadedly engaged with the adjustment nut, such that the mounting bracket can swivel relative to the ball joint base with adjustable tightness.

18. The self-supporting universal monitoring assembly according to claim 1, wherein the secondary sensing device includes at least one selected from a group consisting of a Hall sensor, a GPS positioning module, a contact position sensor, a proximity sensor, a gyroscope, a magnetic rotary encoder, a strain gauge, an accelerometer, a haptic feedback sensor, a LIDAR sensor, and any combination thereof.

19. The self-supporting universal monitoring assembly according to claim 1, wherein the main monitoring device further includes an alarm module, which is configured to be triggered manually or automatically to issue at least one of an audible alarm, a light alarm, and an SOS distress signal.

20. A rollator, comprising the self-supporting universal monitoring assembly according to claim 1, which is detachably mounted to the rollator.
